(19) United States
(12) Patent Application Publication
(10) Pub. No.: US 2017/0112825 A1
(43) Pub. Date: Apr. 27, 2017

(54) METHODS AND COMPOSITIONS FOR REGULATING CONVERSION OF A PRODRUG TO AN ACTIVE PHARMACEUTICAL INGREDIENT

(71) Applicant: Acura Pharmaceuticals, Inc., Palatine, IL (US)

(72) Inventor: Robert Barnett JONES, Summit, NJ (US)

(73) Assignee: Acura Pharmaceuticals, Inc., Palatine, IL (US)

(21) Appl. No.: 15/331,643

(22) Filed: Oct. 21, 2016

Related U.S. Application Data
(60) Provisional application No. 62/245,056, filed on Oct. 22, 2015.

(51) Int. Cl. A61K 31/485; A61K 47/48; A61K 9/00

(52) U.S. Cl. CPC A61K 31/485; A61K 9/0053; A61K 47/48038; A61K 47/48023

(57) ABSTRACT

An abuse deterrent pharmaceutical composition including an abuse deterrent pharmaceutical composition including a prodrug of a pharmaceutically active ingredient and an enzyme inhibitor, wherein the enzyme inhibitor retards conversion of the prodrug to the pharmaceutically active ingredient when the composition is ingested in excess of an intended dosage.

### CROSS-REFERENCE TO RELATED APPLICATIONS

This application claims the benefit of U.S. Provisional Application No. 62/245,056, filed Oct. 22, 2015, the content of which is incorporated herein in its entirety by reference thereto.

### BACKGROUND OF THE INVENTION

The class of drugs exhibiting opium or morphine-like properties is referred to as opioids, or opioid agonists. As agonists, certain drugs are characterized as interacting with stereo specific and saturable binding sites in the brain and other body tissues and organs. Endogenous opioid-like peptides are present in areas of the central nervous system that are presumed to be related to the perception of pain; to movement, mood and behavior; and to the regulation of neuroendocrinological functions. Three classical opioid receptor types, mu (μ, delta (δ), and kappa (κ), have been studied extensively. Each of these receptors has a unique anatomical distribution in the brain, spinal cord, and the periphery. Most of the clinically used opioids are relatively selective for μ receptors, reflecting their similarity to morphine. However, opioid containing drugs that are relatively selective for a particular receptor subtype at standard therapeutic doses will often interact with multiple receptor subtypes when given at sufficiently high doses, leading to possible changes in their pharmacological effect. This is especially true as opioid doses are escalated to overcome tolerance.

The potential for the development of tolerance, physical and/or psychological dependence (i.e., addiction) with repeated opioid use is a characteristic feature of most drugs containing opioid analgesics. The possibility of developing addiction is one of the major concerns in the use of opioids for the management of pain. Another major concern associated with the use of opioids is the diversion of these drugs from a patient in legitimate pain to other individuals (non-patients) for recreational purposes.

Drug abusers and/or addicts typically may take a solid dosage form intended for oral administration containing one or more opioid analgesics and crush, shear, grind, chew, dissolve and/or heat, extract or otherwise tamper with or damage the dosage unit so that a significant portion or even the entire amount of the active drug becomes available for administration by 1) injection, 2) inhalation, and/or 3) oral consumption in amounts exceeding the typical therapeutic dose for such drugs.

There are three basic patterns of behavior leading to opioid abuse. The first involves individuals whose opioid drug use begins in the context of legitimate medical treatment and who obtain their initial drug supplies through prescriptions from appropriately licensed health care providers. Through an insidious process these individuals may ultimately begin seeking prescription drug supplies far exceeding their legitimate medical needs from multiple health care providers and/or pharmacies and/or from illicit sources diverted from otherwise legal drug distribution channels. The second pattern of abuse begins with experimental or “recreational” drug users seeking a “high” with no legitimate medical indication for drugs subject to abuse. A third pattern of abuse involves users who begin in one or another of the preceding ways and ultimately switch to orally administered drugs obtained from organized and legitimate addiction treatment programs.

There are various routes of administration an abuser may commonly employ to abuse an opioid containing drug formulation. The most common methods include 1) parenteral (e.g. intravenous injection), 2) intranasal (e.g., snorting), and 3) repeated oral ingestion of excessive quantities, for example, of orally administered tablets or capsules. One mode of abuse of oral solid drugs involves the extraction of the opioid component from the dosage form by first mixing the dosage form with a suitable solvent (e.g., water), and then subsequently extracting the opioid component from the mixture for use in a solution suitable for intravenous injection of the opioid to achieve a “high.”

Attempts have been made to diminish the abuse potential of orally administered drugs. These attempts generally centered on the inclusion in the oral dosage form of an antagonist which is not orally active but which will substantially block the effects of the drug if one attempts to dissolve the drug and administer it parenterally.

Despite all attempts, the misuse and abuse of pharmaceutical products continues to increase. Clearly there is a growing need for novel and effective methods and compositions to deter abuse of pharmaceutical products (e.g., orally administered pharmaceutical products) including but not limited to immediate release, sustained or extended release and delayed release formulations for drugs subject to abuse. In particular, such methods and compositions would be useful for opioid analgesics, for patients seeking drug therapy, which deter abuse and minimizes or reduces the potential for physical or psychological dependency.

### SUMMARY OF THE INVENTION

According to some embodiments of the invention, an abuse deterrent pharmaceutical composition includes a prodrug of a pharmaceutically active ingredient and an enzyme inhibitor; wherein the enzyme inhibitor retards conversion of the prodrug to the pharmaceutically active ingredient when the composition is ingested in excess of an intended dosage.

In some embodiments the prodrug comprises a pharmaceutically active ingredient modified with a promoiety. In some embodiments, the promoiety is attached to the pharmaceutically active ingredient by an enzymatically cleavable bond. In some embodiments, the prodrug is an ester of the pharmaceutically active ingredient. In some embodiments the promoiety modifies a ketone group of the pharmaceutically active ingredient, while in other embodiments the promoiety modifies a hydroxyl group of the pharmaceutically active ingredient.

In some embodiments the enzyme inhibitor is configured to bind with an enzyme when the composition is ingested in excess of an intended dosage. In other embodiments, the enzyme inhibitor is configured to protect the promoiety from being removed by an enzyme when the composition is ingested in excess of an intended dosage. In some embodiments, the enzyme inhibitor is configured to protect the enzymatically cleavable bond from being cleaved by an enzyme when the composition is ingested in excess of an intended dosage. In some embodiments the enzyme is selected from the group consisting of: CYP2D6, CYP3A4, CYP2D6, CYP2C8, CYP2C19, CYP2D6, and CYP2C9.

In some embodiments, the the pharmaceutically active ingredient comprises a drug susceptible to abuse. In some embodiments the pharmaceutically active ingredient is an opioid.

### DETAILED DESCRIPTION OF THE INVENTION

In some embodiments, formulations of the present invention are designed to block or thwart the effects caused by intentional or unintentional over-ingestion of drug products. Under normal dosing conditions the inventive formulations may allow for the complete and/or bioequivalent oral delivery of the desired drug dose. However when excess doses are ingested, either intentionally or unintentionally, the inventive formulations may work to either slow or block the release and subsequent absorption of the excessive doses. Thus, in the case of intentional over-ingestion where a drug abuser would consume excess doses of an abused drug to experience a euphoric effect, the effect would be significantly reduced for the inventive formulations compared to doses which freely release the excess drug of abuse. In this way, the inventive formulation may work as a deterrent from abusing the inventive formulations for the purpose of achieving the euphoric effect. Yet the patient who uses the invention as directed will receive the desired therapeutic treatment.

In general, and as described in more detail herein, pharmaceutical formulations of the present invention may be designed with one or more components to control release and/or absorption of an active pharmaceutical ingredient. In some embodiments, a pharmaceutical formulation may be designed with an enzyme inhibiting feature. An enzyme inhibiting feature may impact the conversion in vivo of a prodrug of a pharmaceutically active ingredient to the pharmaceutically active ingredient, based on whether the pharmaceutical composition is taken at an appropriate dosage amount or in excess. An enzyme inhibiting feature may be provided by inclusion of one or more enzyme inhibiting and/or blocking ingredients in the pharmaceutical composition. An enzyme inhibiting feature may impact conversion of a prodrug of an active pharmaceutical ingredient to the active pharmaceutical ingredient by binding to an enzyme and/or protecting a promoiety and/or enzymatically cleavable bond of the prodrug, depending on the concentration of the enzymatically inhibiting feature present in the body.

### Components

### Active Pharmaceutical Ingredients

Any drug, therapeutically acceptable drug salt, drug derivative, drug analog, drug homologue, or polymorph can be used in the present invention. Suitable drugs for use with the present invention can be found in the Physician's Desk Reference, 59th Edition, the content of which is hereby incorporated by reference. In one embodiment, the drug is an orally administered drug.

In certain embodiments, drugs susceptible to abuse are used. Drugs commonly susceptible to abuse include psychoactive drugs and analgesics, including but not limited to opioids, opiates, stimulants, tranquilizers, sedatives, anxiolytics, narcotics and drugs that can cause psychological and/or physical dependence. In one embodiment, the drug for use in the present invention can include amphetamines, amphetamine-like compounds, benzodiazepines, and methyl phenidate or combinations thereof. In another embodiment, the present invention can include any of the resolved isomers of the drugs described herein, and/or salts thereof.

A drug for use in the present invention which can be susceptible to abuse can be one or more of the following: alfentanil, amphetamines, buprenorphine, butorphanol, carfentanil, codeine, dezocine, diacetylmorphine, dihydrocodeine, dihydromorphine, diphenoxylate, diprenorphine, etorphine, fentanyl, hydrocodone, hydromorphone, β-hydroxy-3-methylfentanyl, levo-a-acetylmethadol, levorphanol, lofentanil, meperidine, methadone, methylphenidate, morphine, nalbuphine, nalmefene, oxycodone, oxymorphone, pentazocine, pethidine, propoxyphene, remifentanil, sufentanil, tilidine, and tramodol, salts, derivatives, analogs, homologues, polymorphs thereof, and mixtures of any of the foregoing.

In another embodiment a drug for use with the present invention which can be susceptible to abuse includes one or more of the following: dextromethorphan (3-Methoxy-17-methy-9a, 13a, 1 4a-morphinan hydrobromide monohydrate), N-{1-[2-(4-ethyl-5-oxo-2-tetrazolin-1-yl)-ethyl]-4-methoxymethyl-4-piperidyl} propionanilide (alfentanil), 5,5-diallyl barbituric acid (allobarbital), allylprodine, alpha-prodine, 8-chloro-1-methyl-6-phenyl-4H-[1,2,4]triazolo[4,3-a][1,4]-benzodiazepine (alprazolam), 2-diethylaminopropiophenone (amfepramone), (±)-α-methyl phenethylamine (amphetamine), 2-(α-methylphenethyl-amino)-2-phenyl acetonitrile (amphetaminil), 5-ethyl-5-isopentyl barbituric acid (amobarbital), anileridine, apocodeine, 5,5-diethyl barbituric acid (barbital), benzylmorphine, bezitramide, 7-bromo-5-(2-pyridyl)-1H-1,4-benzodiazepin-2(3H)-one (bromazepam), 2-bromo-4-(2-chlorophenyl)-9-methyl-6H-thieno[3,2-f][1,2,4]-triazolo[4,3-a][1,4]diazepine (brotizolam), 17-cyclopropylmethyl-4,5α-epoxy-7α[(S)-1-hydroxy-1,2,2-trimethylpropyl]-6-methoxy-6,14-endo-ethanomorphinan-3-ol (buprenorphine), 5-butyl-5-ethyl barbituric acid (butobarbital), butorphanol, (7-chloro-1,3-dihydro-1-methyl-2-oxo-5-phenyl-2H-1,4-benzodiazepin-3-yl)-dimethyl carbamate (camazepam), (1S,2S)-2-amino-1-phenyl-1-propanol (cathine/D-norpseudoephedrine), 7-chloro-N-methyl-5-phenyl-3H-1,4-benzodiazepin-2-ylamine-4 oxide (chlordiazepoxide), 7-chloro-l-methyl-5-phenyl-1H-1,5-benzodiazepine-2,4(3H,5H)-dione (clobazam), 5-(2-chlorophenyl)-7-nitro-1H-1,4-benzodiazepin-2(3H)-one (clonazepam), clonitazene, 7-chloro-2,3-dihydro-2-oxo-5-phenyl-1H-1,4-benzodiazepine-3-carboxylic acid (clorazepate), 5-(2-chlorophenyl)-7-ethyl-1-methyl-1H-thieno[2,3-e][1,4]-diazepin-2(3H)-one (clotiazepam), 10-chloro-11b-(2-chlorophenyl)-2,3,7,11b-tetrahydrooxazolo[3,2-d][1,4]benzodiazepin-6(5H)-one (cloxazolam), (−)-methyl-[3β-benzoyloxy-2β(1αH,5αH)-tropane carboxylate (cocaine), 4,5α-epoxy-3-methoxy-17-methyl-7-morphinen-6α-ol (codeine), 5-(1-cyclohexenyl)-5-ethyl barbituric acid (cyclobarbital), cyclorphan, cyprenorphine, 7-chloro-5-(2-chlorophenyl)-1H-1,4-benzodiazepin-2(3H)-one (delorazepam), desomorphine, dextromoramide, (+)-(1-benzyl-3-dimethylamino-2-methyl-1-phenylpropyl) propionate (dextropropoxyphene), dezocine, diampromide, diamorphone, 7-chloro-1-methyl-5-phenyl-1H-1,4-benzodiazepin-2(3H)-one (diazepam), 4,5α-epoxy-3-methoxy-17-methyl-6α-morphinanol (dihydrocodeine), 4,5α-epoxy-17-methyl-3,6a-morphinandiol (dihydromorphine), dimenoxadol, dimephetamol [sic-Tr.Ed.], dimethyl thiambutene, dioxaphetyl butyrate, dipipanone, (6aR,10aR)-6,6,9-trimethyl-3-pentyl-6a,7,8,10a-tetrahydro-6H-benzo[c]chromen-1-ol (dronabinol), eptazocine, 8-chloro-6-phenyl-4H-[1,2,4]triazolo[4,3-a][1,4]benzodiazepine (estazolam), ethoheptazine, ethyl methyl thiambutene, ethyl-[7-chloro-5-(2-fluorophenyl)-2,3-dihydro-2-oxo-1H-1,4-benzodiazepin-3-carboxylate] (ethyl loflazepate), 4,5α-epoxy-3-ethoxy-17-methyl-7-morphinen-6α-ol (ethylmorphine), etonitrazene, 4,5α-epoxy-7α-(1-hydroxy-1-methylbutyl)-6-methoxy-17-methyl-6,14-endo-etheno-morphinan-3-ol (etorphine), N-ethyl-3-phenyl-8,9,10-trinorbornan-2-ylamine (fencamfamine), 7-[2-(α-methylphenethylamino)-ethyl] theophylline (fenethylline), 3-(α-methylphenethylamino) propionitrile (fenproporex), N-(1-phenethyl-4-piperidyl) propionanilide (fentanyl), 7-chloro-5-(2-fluorophenyl)-1-methyl-1H-1,4-benzodiazepin-2(3H)-one (fludiazepam), 5-(2-fluorophenyl)-1-methyl-7-nitro-1H-1,4-benzodiazepin-2-(3H)-one (flunitrazepam), 7-chloro-1-(2-diethylaminoethyl)-5-(2-fluorophenyl)-1H-1,4-benzodiazepin-2(3H)-one (flurazepam), 7-chloro-5-phenyl-1-(2,2,2-trifluoroethyl)-1H-1,4-benzodiazepin-2(3H)-one (halazepam), 10-bromo-11b-(2-fluorophenyl)-2,3,7,11b-tetrahydro[1,3]oxazolo[3,2-d][1,4]benzodiazepin-6(5H)-one (haloxazolam), heroin, 4,5α-epoxy-3-methoxy-17-methyl-6-morphinanone (hydrocodone), 4,5α-epoxy-3-hydroxy-17-methyl-6-morphinanone (hydromorphone), hydroxypethidine, isomethadone, hydroxymethyl morphinan, 11-chloro-8,12b-dihydro-2,8-dimethyl-12b-phenyl-4H-[1,3]oxazino[3,2-d][1,4]benzodiazepin-4,7(6H)-dione (ketazolam), 1-[4-(3-hydroxyphenyl)-1-methyl-4-piperidyl]-1-propanone (ketobemidone), (3S,6S)-6-dimethylamino-4,4-diphenylheptan-3-yl acetate (levacetylmethadol (LAAM)), (−)-6-dimethylamino-4,4-diphenyl-3-heptanone (levomethadone), (−)-17-methyl-3-morphinanol (levorphanol), levophenacyl morphan, lofentanil, 6-(2-chlorophenyl)-2-(4-methyl-1-piperazinylmethylene)-8-nitro-2H-imidazo[1,2a][1,4]benzodiazepin-1(4H)-one (loprazolam), 7-chloro-5-(2-chlorophenyl)-3-hydroxy-1H-1,4-benzodiazepin-2(3H)-one (lorazepam), 7-chloro-5-(2-chlorophenyl)-3-hydroxy-1-methyl-1H-1,4-benzodiazepin-2(3H)-one (lormetazepam), 5-(4-chlorophenyl)-2,5-dihydro-3H-imidazo[2,1-a]isoindol-5-ol (mazindol), 7-chloro-2,3-dihydro-l-methyl-5-phenyl-1H-1,4-benzodiazepine (medazepam), N-(3-chloropropyl)-α-methylphenetylamine (mefenorex), meperidine, 2-methyl-2-propyl trimethylene dicarbamate (meprobamate), meptazinol, metazocine, methylmorphine, N,α-dimethylphenethylamine (methamphetamine), (±)-6-dimethylamino-4,4-diphenyl-3-heptanone (methadone), 2-methyl-3-o-tolyl-4(3H)-quinazolinone (methaqualone), methyl-[2-phenyl-2-(2-piperidyl)acetate] (methyl phenidate), 5-ethyl-1-methyl-5-phenyl barbituric acid (methyl phenobarbital), 3,3-diethyl-5-methyl-2,4-piperidinedione (methyprylon), metopon, 8-chloro-6-(2-fluorophenyl)-1-methyl-4H-imidazo[1,5-a][1,4]benzodiazepine (midazolam), 2-(benzhydrylsulfinyl) acetamide (modafinil), 4,5α-epoxy-17-methyl-7-morphinene-3,6α-diol (morphine), myrophine, (±)-trans-3-(1,1-dimethylheptyl)-7,8,10,10α-tetrahydro-1-hydroxy-6,6-dimethyl-6H-dibenzo[b,d]pyran-9(6αH)-one (nabilone), nalbuphen, nalorphine, narceine, nicomorphine, 1-methyl-7-nitro-5-phenyl-1H-1,4-benzodiazepin-2(3H)-one (nimetazepam), 7-nitro-5-phenyl-1H-1,4-benzodiazepin-2(3H)-one (nitrazepam), 7-chloro-5-phenyl-1H-1,4-benzodiazepin-2-(3H)-one (nordazepam), norlevorphanol, 6-dimethylamino-4,4-diphenyl-3-hexanone (normethadone), normorphine, norpipanone, the coagulated juice of the plants belonging to the species Papaver somniferum (opium), 7-chloro-3-hydroxy-5-phenyl-1H-1,4-benzodiazepin-2-(3H)-one (oxazepam), (cis-trans)-10-chloro-2,3,7,1 b-tetrahydro-2-methyl-11b-phenyloxazolo[3,2-d][1,4]benzodiazepin-6-(5H)-one (oxazolam), 4,5a-epoxy-14-hydroxy-3-methoxy-17-methyl-6-morphinanone (oxycodone), oxymorphone, plants and plant parts of the plants belonging to the species Papaver somniferum (including the subspecies setigerum) (Papaver somniferum), papaveretum, 2-imino-5-phenyl-4-oxazolidinone (pemoline), 1,2,3,4,5,6-hexahydro-6,11-dimethyl-3-(3-methyl-2-butenyl)-2,6-methano-3-benzazocin-8-ol (pentazocine), 5-ethyl-5-(1-methylbutyl) barbituric acid (pentobarbital), ethyl-(1-methyl-4-phenyl-4-piperidine-carboxylate) (pethidine), phenadoxone, phenomorphan, phenazocine, phenoperidine, piminodine, pholcodeine, 3-methyl-2-phenyl morpholine (phenmetrazine), 5-ethyl-5-phenyl barbituric acid (phenobarbital), α,α-dimethyl phenethylamine (phentermine), 7-chloro-5-phenyl-1-(2-propinyl)-1H-1,4-benzodiazepin-2(3H)-one (pinazepam), α-(2-piperidyl)benzhydryl alcohol (pipradol), 1′-(3-cyano-3,3-diphenylpropyl)[1,4′-bipiperidine]-4′-carboxamide (piritramide), 7-chloro-1-(cyclopropylmethyl)-5-phenyl-1H-1,4-benzodiazepin-2(3H)-one (prazepam), profadol, proheptazine, promedol, properidine, propoxyphene, N-(1-methyl-2-piperidinoethyl)-N-(2-pyridyl) propionamide, methyl-{3-[4-methoxycarbonyl-4-(N-phenylpropaneamido)piperidino]propanoate} (remifentanil), 5-sec.-butyl-5-ethyl barbituric acid (secbutabarbital), 5-allyl-5-(1-methylbutyl) barbituric acid (secobarbital), N-{4-methoxymethyl-1-[2-(2-thienyl)ethyl]-4-piperidyl} propionanilide (sufentanil), 7-chloro-2-hydroxy-methyl-5-phenyl-1H-1,4-benzodiazepin-2-(3H)-one (temazepam), 7-chloro-5-(1-cyclohexenyl)-1-methyl-1H-1,4-benzodiazepin-2(3H)-one (tetrazepam), ethyl-(2-dimethylamino-1-phenyl-3-cyclohexane-1-carboxylate) (tilidine (cis and trans)), tramadol, 8-chloro-6-(2-chlorophenyl)-1-methyl-4H-[1,2,4]triazolo[4,3-a][1,4]benzodiazepine (triazolam), 5-(1-methylbutyl)-5-vinyl barbituric acid (vinylbital), (1R*,2R*)-3-(3-dimethylamino-1-ethyl-2-methyl-propyl) phenol, (1R,2R,4S)-2-[dimethylamino)methyl-4-(p-fluorobenzyloxy)-1-(m-methoxyphenyl) cyclohexanol, each optionally in the form of corresponding stereoisomeric compounds as well as corresponding derivatives, especially esters or ethers, and all being physiologically compatible compounds, especially salts and solvates.

In one embodiment, a pharmaceutical composition of the present invention includes one or more opioids such as hydrocodone, hydromorphone, morphine and oxycodone and/or salts thereof, as the therapeutically active ingredient. Typically when processed into a suitable dosage form, as described in more detail below, the drug can be present in such dosage forms in an amount normally prescribed, typically about 0.5 to about 25 percent on a dry weight basis, based on the total weight of the formulation.

With respect to analgesics in unit dose form, such drugs may be present in a pharmaceutically acceptable amount; standard doses of such drugs are generally known in the art and are disclosed, for example, in the United States Pharmacopeia and National Formulary (USP 36-NF 31). Rockville, Md.: United States Pharmacopeia Convention; 2013, which is incorporated by reference herein in its entirety. In some embodiments, such drugs may be present in an amount of about 5, 25, 50, 75, 100, 125, 150, 175 or 200 mg. In some embodiments, the drug can be present in an amount from about 5 to about 500 mg or about 5 to about 200 mg. In some embodiments, a dosage form contains an appropriate amount of drug to provide a therapeutic effect.

### Prodrug of an Active Pharmaceutical Ingredients

A “prodrug” refers to an active pharmaceutical ingredient that is modified by a chemical group (a “promoiety”), which provides one or more desired beneficial properties, such as increased solubility, permeability, or ease of administration. A prodrug may, for instance, be bioavailable by oral administration whereas the active pharmaceutical ingredient is not. The prodrug may also have improved solubility in pharmaceutical compositions over the parent drug. An example, without limitation, of a prodrug would be a compound described herein, which is administered as an ester but which then is enzymatically hydrolyzed to the carboxylic acid, the active entity. A further example of a prodrug might be an active pharmaceutical ingredient modified by a short peptide (polyaminoacid) bonded to an acid group where the peptide is metabolized to reveal the active moiety. The prodrug of an active pharmaceutical ingredient may be converted into the active pharmaceutical ingredient in vivo, for example, by an enzymatic or chemical process that removes the promoiety. Prodrugs which are converted to active forms through other mechanisms in vivo are also included.

Prodrugs of a pharmaceutically active ingredient can be prepared in situ during the final isolation and purification of the pharmaceutically active ingredients, or by separately reacting the purified pharmaceutically active ingredient in its free acid form or hydroxyl with a suitable esterifying agent. Hydroxyl groups can be converted into esters via treatment with a carboxylic acid. Examples of prodrug moieties include substituted and unsubstituted, branched or unbranched lower alkyl ester moieties, lower alkenyl esters, di-lower alkyl-amino lower-alkyl esters, acylamino lower alkyl esters, acyloxy lower alkyl esters, aryl esters, aryl-lower alkyl esters, substituted (e.g., with methyl, halo, or methoxy substituents) aryl and aryl-lower alkyl esters, amides, lower-alkyl amides, di-lower alkyl amides, and hydroxy amides. In one embodiment, the prodrug is an orally administered prodrug of an active pharmaceutical ingredient.

Any prodrug of an active pharmaceutical agent can be used in the present invention, alone or in combination with any active pharmaceutical ingredient described herein. In some embodiments, a prodrug of an active pharmaceutical ingredient for use with the present invention may be a prodrug of an opioid. In some embodiments a prodrug of an opioid may be one or more of the following: morphine, codeine, hydrocodone, oxycodone, methadone, tramadol, fentanyl, hydromorphone, or oxymorphone. In some embodiments a prodrug. In some embodiments, a prodrug of an opioid may be a prodrug of morphine, a prodrug of codeine, a prodrug of hydrocodone, a prodrug of oxycodone, a prodrug of methadone, a prodrug of tramadol, a prodrug of fentanyl, a prodrug of hydromorphone, or a prodrug of oxymorphone.

A prodrug of an active pharmaceutical ingredient may comprise an active pharmaceutical ingredient modified by any promoiety that is suitable for providing the prodrug with a desired effect (e.g. increased bioavailability or inactivity of the active pharmaceutical ingredient) and is removable in vivo. In some embodiments a prodrug of an active pharmaceutical ingredient comprises an active pharmaceutical ingredient modified by a hydroxybenzoic acid, a benzoic acid, or an aminobenzoic acid. In some embodiments a prodrug of an active pharmaceutical ingredient comprises an active pharmaceutical ingredient modified by benzoic acid, salicylic acid, aspirin, 3-hydroxy-benzoic acid, 4-hydroxy-benzoic acid, 6-methylsalicylic acid, o-cresotinic acid, anacardic acid, o-thymotic acid, diflunisal, p-anisic acid, 2,3-dihydroxy-benzoic acid (2,3-DHB), a-resorcylic acid, protocatechuic acid, gentisic acid, piperonylic acid, 3-methoxy-salicylic acid, 4-methoxy-salicylic acid, vanillic acid, isovanillic acid, veratric acid, 3,5-dimethoxy-benzoic acid, gallic acid, 2,3,4-trihydroxy-benzoic acid, 2,3,6-trihydroxy-benzoic acid, 2,4,5-trihydroxy-benzoic acid, 3-o-methylgallic acid (3-OMGA), 4-o-methylgallic acid (4-OMGA), syringic acid, 3,4,5-trimethoxy-benzoic acid, anthranillic acid, 3-aminobenzoic acid, 4,5-dimethyl-anthranilic acid, N-methylanthranilic acid, fenamic acid, tolfenamic acid, mefenamic acid, flufenamic acid, 2,4-diaminobenzoic acid (2,4-DABA), N-acetylanthranilic acid, 2-acetylamino-4-aminobenzoic acid, 2,4-diacetylamino-benzoic acid, 4-aminosalicylic acid, 3-hydroxyanthranilic acid, 3-methoxyanthranilic acid, or a combination thereof.

In some embodiments a prodrug of an active pharmaceutical ingredient comprising a ketone. In some embodiments a prodrug of an active pharmaceutical ingredient comprising a ketone is selected from a prodrug of oxycodone, a prodrug of hydromorphone, a prodrug of hydrocodone, a prodrug of methadone, a prodrug of fentanyl, and a prodrug of oxymorphone. In some embodiments the prodrug of an active pharmaceutical ingredient comprising a ketone is modified at the ketone oxygen. In some embodiments a prodrug of hydromorphone is referred to as KP511 (KemPharm®). In some embodiments a prodrug of oxycodone is referred to as KP606 (KemPharm®).

In some embodiments a prodrug of hydromorphone comprises Formula 1 (also referred to as PF329, Signature Therapeutics®):

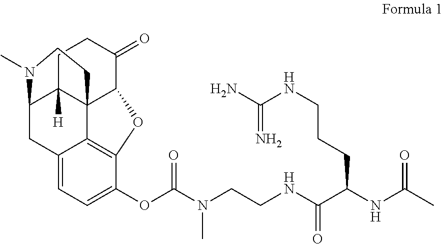 [SMILES: CC(=O)N[C@H](CCCNC(=N)N)C(=O)NCCN(C)C(=O)Oc1ccc2c3c1O[C@H]1C(=O)CC[C@H]4[C@@H](C2)N(C)CC[C@]314]

In some embodiments a prodrug of hydrocodone comprises Formula 2 (also referred to as KP201, KemPharm®).

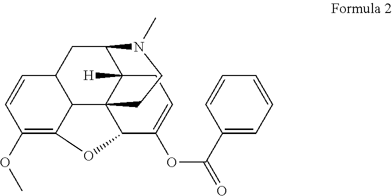

In some embodiments a prodrug of an active pharmaceutical ingredient comprising a hydroxyl. In some embodiments a prodrug of an active pharmaceutical ingredient comprising a hydroxyl is selected from a prodrug of oxycodone,a prodrug of hydromorphone, a prodrug of morphine, a prodrug of codeine, a prodrug of tramadol, and a prodrug of oxymorphone. In some embodiments the prodrug of an active pharmaceutical ingredient comprising a hydroxyl is modified at the hydroxyl oxygen. For example, in some embodiments a prodrug of oxycodone comprises Formula 3 (also referred to as PF614, Signature Therapeutics®):

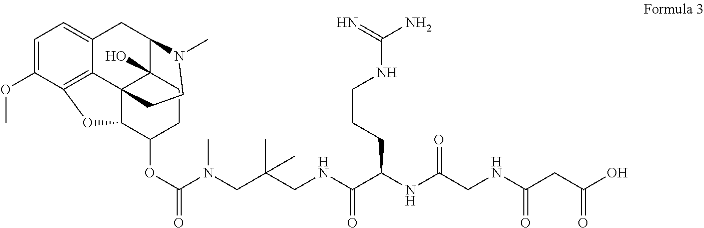 [SMILES: COc1ccc2c3c1O[C@H]1C(OC(=O)N(C)CC(C)(C)CNC(=O)[C@@H](CCCNC(=N)N)NC(=O)CNC(=O)CC(=O)O)CC[C@@]4(O)[C@@H](C2)N(C)CC[C@]314]

In some embodiments, a prodrug of an active pharmaceutical ingredient for use with the present invention may be a prodrug disclosed in U.S. Pat. No. 9,095,627, U.S. Pat. No. 9,040,032, U.S. Pat. No. 8,685,916, U.S. Pat. No. 8,461,137, US Patent Publication No. 20130210700, US Patent Publication No. 20130123292, US Patent Publication No. 20120230916, US Patent Publication No. 20110262360, US Patent Publication No. 20110262355, US Patent Publication No. 20110245288, US Patent Publication No. 20110105381, US Patent Publication No. 20100267614, US Patent Publication No. 20090192095, US Patent Publication No. 20090156814, US Patent Publication No. 20080076789, each of which is hereby incorporated by reference.

### Enzyme Inhibiting Component

In some embodiments, pharmaceutical compositions of the present invention include one or more components which inhibit conversion of the prodrug of the active pharmaceutical ingredient to the active pharmaceutical ingredient in vivo. In some embodiments the enzyme inhibiting component is an enzyme inhibiting component. In some embodiments the enzyme inhibiting component may be a protecting group. For example, the enzyme inhibiting component may inhibit a chemical reaction that would remove a promoiety from the prodrug of the pharmaceutically active ingredient (e.g. the enzyme inhibiting compenent protects the promoiety).

In some embodiments, an enzyme inhibiting component is configured to bind with one or more enzymes involved in conversion of the prodrug to the active pharmaceutical ingredient, as a function of the amount of pharmaceutical ingredient ingested: when the pharmaceutical composition is ingested in an appropriate dosage amount, the enzyme inhibiting agent is not present in an amount sufficient to affect the pharmacokinetics of the metabolism of the prodrug, and one or more enzymes metabolize the prodrug to the active pharmaceutical ingredient; when the pharmaceutical component is ingested in an excess amount, the enzyme inhibiting agent is present in an amount sufficient to bind enough enzyme to affect the pharmacokinetics of the metabolism of the prodrug, thereby preventing the prodrug from being converted by the enzyme to the active pharmaceutical ingredient. In some embodiments the enzyme inhibitor reversibly binds to the enzyme, while in other embodiments the enzyme inhibitor irreversibly binds to the enzyme.

In some embodiments the enzyme inhibiting component inhibits the activity of one or more digestive enzymes or liver enzymes. Digestive enzymes include enzymes secreted by the salivary glands, the stoch, the pancrease, the small intestine, and the large intestine. Digestive enzymes include proteases, lipases, amylases, and nucleases. Digestive enzymes include pepsin, hydrochloric acid, gastric lipase, lingual lipase, trypsin, chymotrypsin, carboxypeptidase, pacreatic lipase, sterol esterase, phospholipase, pancreatic amylase, erepsin, maltase, lactase, and sucrase. Liver enzymes include cytochrome P450 enzymes. In some embodiments the enzyme inhibiting component inhibits the activity of one or more cytochrome P450 enzymes selected from the group consisting of CYP2D6, CYP3A4, CYP2D6, CYP2C8, CYP2C19, CYP2D6, and CYP2C9 on the prodrug. In some embodiments, the enzyme inhibiting component inhibits the metabolism of the prodrug by binding with one or more enzymes selected from the group consisting of: CYP2D6, CYP3A4, CYP2D6, CYP2C8, CYP2C19, CYP2D6, and CYP2C9. In some embodiments, the enzyme inhibiting component inhibits metabolism of the prodrug by protecting the prodrug from binding with one or more enzymes selected from the group consisting of CYP2D6, CYP3A4, CYP2D6, CYP2C8, CYP2C19, CYP2D6, and CYP2C9.

Examples of suitable enzyme inhibiting components include amylase inhibitors, trypsin inhibitors, buproprion, terbinafine, protease inhibitors, ritonavir, and selective serotonin reuptake inhibitors (SSRIs), including fluoxetine. In some embodiments an enzyme inhibiting component may be an active pharmaceutical ingredient.

Amylase inhibitors include alphaAl-1, alphaAl-2, arcelin-5, bean amylase inhibitors, Calorex, Fabaceae (family), phaseolamin, Phaseolus vulgaris extract, starch blockers, Starchex, wheat amylase inhibitor, wheat proteinaceous alpha-amylase inhibitors (alpha-AIs), and white kidney bean extract.

Trypsin inhibitors include derivatives from a variety of animal or vegetable sources: for example, soybean, corn, lima and other beans, squash, sunflower, bovine and other animal pancreas and lung, chicken and turkey egg white, soy-based infant formula, and mammalian blood. Trypsin inhibitors can also be of microbial origin. A trypsin inhibitor can also be an arginine or lysine mimic or other synthetic compound: for example arylguanidine, benzamidine, 3,4-dichloroisocoumarin, diisopropylfluorophosphate, gabexate mesylate, phenylmethanesulfonyl fluoride, or substituted versions or analogs thereof. In certain embodiments, trypsin inhibitors comprise a covalently modifiable group, such as a chloroketone moiety, an aldehyde moiety, or an epoxide moiety. Other examples of trypsin inhibitors are aprotinin, camostat and pentamidine.

In some embodiments, an enzyme inhibiting component (e.g. a protecting group) is configured to protect the promoiety from being cleaved from the prodrug, as a function of the amount of pharmaceutical ingredient ingested: when the pharmaceutical composition is ingested in an appropriate dosage amount, the enzyme inhibiting agent is not present in an amount sufficient to affect the pharmacokinetics of the metabolism of the prodrug, and one or more enzymes metabolize the prodrug to the active pharmaceutical ingredient; when the pharmaceutical component is ingested in an excess amount, the enzyme inhibiting agent is present in an amount sufficient to bind enough enzyme to affect the pharmacokinetics of the metabolism of the prodrug, thereby preventing the prodrug from being converted by the enzyme to the active pharmaceutical ingredient. In some such embodiments, the enzyme inhibiting component is covalently bound to the prodrug and, in some embodiments, may be the promoiey itself.

In some embodiments, an enzyme inhibiting component is included in the pharmaceutical composition in an amount of about 1 wt % to about 50 wt %; about 1 wt % to about 48 wt %; about 1 wt % to about 46 wt %; about 1 wt % to about 44 wt %; about 1 wt % to about 42 wt %; about 1 wt % to about 40 wt %; about 2 wt % to about 38 wt %; about 4 wt % to about 36 wt %; about 6 wt % to about 34 wt %; about 8 wt % to about 32 wt %; about 10 wt % to about 30 wt %; about 12 wt % to about 28 wt %; about 14 wt % to about 26 wt %; about 16 wt % to about 24 wt %; about 18 wt % to about 22 wt %; about 1 wt %; about 2 wt %; about 4 wt %; about 6 wt %; about 8 wt %; about 10 wt %; about 12 wt %; about 14 wt %; about 16 wt %; about 18 wt %; about 20 wt %; about 22 wt %; about 24 wt %; about 26 wt %; about 28 wt %; about 30 wt %; about 32 wt %; about 34 wt %; about 36 wt %; about 38 wt %; about 40 wt %; about 42 wt %; about 44 wt %; about 46 wt %; about 48 wt %; or about 50 wt %.

### Additional Ingredients

The present invention can also optionally include other ingredients to enhance dosage form manufacture from a pharmaceutical composition of the present invention and/or alter the release profile of a dosage form including a pharmaceutical composition of the present invention.

Some embodiments of the present invention include one or more pharmaceutically acceptable fillers/diluents. In one embodiment, Avicel PH (Microcrystalline cellulose) is a filler used in the formulation. The Avicel PH can have an average particle size ranging from 20 to about 200 μm, preferably about 100 The density can range from about 1.512 to about 1.668 g/cm³. The Avicel PH should have molecular weight of about 36,000. Avicel PH effectiveness is optimal when it is present in an amount of from about 10 to 65 percent, by weight on a solid basis, of the formulation. Typical fillers can be present in amounts from 10 to 65 percent by weight on a dry weight basis of the total composition. Other ingredients can include sugars and/or polyols. Lactose having a particle size of about 20 to about 400 microns and a density of about 0.3 to about 0.9 g/ml can also be included.

In some embodiments of the invention, the fillers which can be present at about 10 to 65 percent by weight on a dry weight basis, also function as binders in that they not only impart cohesive properties to the material within the formulation, but can also increase the bulk weight of a directly compressible formulation (as described below) to achieve an acceptable formulation weight for direct compression. In some embodiments, additional fillers need not provide the same level of cohesive properties as the binders selected, but can be capable of contributing to formulation homogeneity and resist segregation from the formulation once blended. Further, preferred fillers do not have a detrimental effect on the flowability of the composition or dissolution profile of the formed tablets.

In one embodiment, the present invention can include one or more pharmaceutically acceptable disintegrants. Such disintegrants are known to a skilled artisan. In the present invention, disintegrants can include, but are not limited to, sodium starch glycolate (Explotab®) having a particle size of about 104 microns and a density of about 0.756 g/ml, starch (e.g., Starch 21) having a particle size of about 2 to about 32 microns and a density of about 0.462 g/ml, Crospovidone® having a particle size of about 400 microns and a density of about 1.22 g/ml, and croscarmellose sodium (Ac-Di-Sol) having a particle size of about 37 to about 73.7 microns and a density of about 0.529 g/ml. The disintegrant selected should contribute to the compressibility, flowability and homogeneity of the formulation. Further the disintegrant can minimize segregation and provide an immediate release profile to the formulation. In some embodiments, the disintegrant(s) are present in an amount from about 2 to about 25 percent by weight on a solid basis of the directly compressible formulation. Furthermore, antacids added to the formulations may aid in tablet disintegration when the tablet is introduced to a low pH environment through the effervescense of the antacid ingredient, thus potentially reducing the requirement for additional disintegrants.

In one embodiment, the present invention can include one or more pharmaceutically acceptable glidants, including but not limited to colloidal silicon dioxide. In one embodiment, colloidal silicon dioxide (Cab-O-Sil®) having a density of about 0.029 to about 0.040 g/ml can be used to improve the flow characteristics of the formulation. Such glidants can be provided in an amount of from about 0.1 to about 1 percent by weight of the formulation on a solid basis. It will be understood, based on this invention, however, that while colloidal silicon dioxide is one particular glidant, other glidants having similar properties which are known or to be developed could be used provided they are compatible with other excipients and the active ingredient in the formulation and which do not significantly affect the flowability, homogeneity and compressibility of the formulation.

In one embodiment, the present invention can include one or more pharmaceutically acceptable lubricants, including but not limited to magnesium stearate. In one embodiment, the magnesium stearate has a particle size of about 450 to about 550 microns and a density of about 1.00 to about 1.80 g/ml. In one embodiment, magnesium stearate can contribute to reducing friction between a die wall and a pharmaceutical composition of the present invention during compression and can ease the ejection of the tablets, thereby facilitating processing. In some embodiments, the lubricant resists adhesion to punches and dies and/or aid in the flow of the powder in a hopper and/or into a die. In an embodiment of the present invention, magnesium stearate having a particle size of from about 5 to about 50 microns and a density of from about 0.1 to about 1.1 g/ml is used in a pharmaceutical composition. In certain embodiments, a lubricant should make up from about 0.1 to about 2 percent by weight of the formulation on a solid basis. Suitable lubricants are stable and do not polymerize within the formulation once combined. Other lubricants known in the art or to be developed which exhibit acceptable or comparable properties include stearic acid, hydrogenated oils, sodium stearyl fumarate, polyethylene glycols, and Lubritab®.

In certain embodiments, the most important criteria for selection of the excipients are that the excipients should achieve good content uniformity and release the active ingredient as desired. The excipients, by having excellent binding properties, and homogeneity, as well as good compressibility, cohesiveness and flowability in blended form, minimize segregation of powders in the hopper during compression.

### Controlled Conversion Dosage Forms

As described herein, pharmaceutical formulations of the present invention may be formulated to slow or block the conversion to and subsequent absorption of excessive doses of an active pharmaceutical ingredient. In some embodiments, a pharmaceutical formulation may be designed with an enzyme inhibiting feature. An enzyme inhibiting feature may impact conversion of a prodrug to an active ingredient by binding with an enzyme that is a part of the enzyme pathway of the prodrug, based on whether the pharmaceutical composition is taken in an appropriate dosage amount or in excess. An enzyme inhibiting feature may impact conversion of a prodrug to an active ingredient by protecting the promoiety from being removed by an enzyme, based on whether the pharmaceutical composition is taken in an appropriate dosage amount or in excess. An enzyme inhibiting feature may be provided by inclusion of one or more enzyme inhibiting ingredients in the pharmaceutical composition, and/or, an enzyme inhibiting feature may be provided by inclusion of promoiety protection group in the pharmaceutical composition. In some embodiments an enzyme inhibitor may be a pharmaceutically active ingredient.

In some embodiments, the pharmaceutical composition may be formulated such that when the composition is taken in appropriate amounts, an enzyme inhibiting feature has minimal impact (i.e., the rate of conversion of prodrug to active ingredient is not substantially modified or is maintained at a desirable level), thereby allowing conversion of the prodrug to the active pharmaceutical ingredient. However, when the pharmaceutical composition is ingested in excess, in some embodiments the composition is formulated such that the enzyme inhibiting feature has a significant or maximal impact (i.e., the rate of conversion of prodrug to active ingredient is retarded), thereby thwarting conversion of the prodrug to the active pharmaceutical ingredient.

Under conditions where excess doses are ingested, intentionally or unintentionally, (i.e., three tablets or greater), the quantity of enzyme inhibitor from over-ingestion may now be sufficient to retard conversion of the prodrug to the active pharmaceutical ingredient. For example, under conditions where excess doses are ingested, an enzyme inhibitor (e.g. enzyme inhibitor) may be present in quantities sufficient to drive enzyme reactivity in favor of the enzyme inhibitor and away from the prodrug, thereby retarding conversion of the prodrug to the pharmaceutically active ingredient. In another example, under conditions where excess doses are ingested, an enzyme inhibitor may be present in quantities sufficient to block the metabolic pathway of the prodrug by protecting the promoiety of the prodrug or otherwise blocking the prodrug from binding with one or more enzyme enzymes.

In some embodiments, a pharmaceutical composition may be prepared by intimately mixing the prodrug of an active pharmaceutical ingredient with an enzyme inhibitor by any suitable process (i.e. dry or wet granulation, hot melt extrusion, etc.) such that a particulate matrix is formed in a particulate form.

Suitable formulations and dosage forms of the present invention include but are not limited to powders, caplets, pills, suppositories, gels, soft gelatin capsules, capsules and compressed tablets manufactured from a pharmaceutical composition of the present invention. The dosage forms can be any shape, including regular or irregular shape depending upon the needs of the artisan.

Compressed tablets including the pharmaceutical compositions of the present invention can be direct compression tablets or non-direct compression tablets. In one embodiment, a dosage form of the present invention can be made by wet granulation, and/or dry granulation (e.g., slugging or roller compaction). The method of preparation and type of excipients are selected to give the tablet formulation desired physical characteristics that allow for the rapid compression of the tablets. After compression, the tablets must have a number of additional attributes such as appearance, hardness, disintegrating ability, and an acceptable dissolution profile.

Choice of fillers and other excipients typically depend on the chemical and physical properties of the drug, behavior of the mixture during processing, and the properties of the final tablets. Adjustment of such parameters is understood to be within the general understanding of one skilled in the relevant art. Suitable fillers and excipients are described in more detail above.

The manufacture of a dosage form of the present invention can involve direct compression and wet and dry granulation methods, including slugging and roller compaction.

In some embodiments, one or more components may be sequestered, as described in U.S. Patent Application Publication No. 2012/0202839 which is incorporated by reference herein in its entirety.

The present invention can be used to manufacture immediate release, and controlled drug release formulations. Controlled release formulations can include delayed release, bi-modal and tri-modal release, extended and sustained release oral solid dosage preparations.

As used herein, the term “about” is understood to mean +10% of the value referenced. For example, “about 45%” is understood to literally mean 40.5% to 49.5%.

As used herein, the term “bioequivalence” is understood to mean one or more of Cₘₐₓ, Tₘₐₓ, or area under the concentration curve “AUC” of a drug is within 75% to 120% of the same marker for a referenced drug.

It will be appreciated by persons skilled in the art that numerous variations and/or modifications may be made to the invention shown in the specific embodiments without departing from the spirit and scope of the invention as broadly described. Further, each and every reference cited above is hereby incorporated by reference as if fully set forth herein.

What is claimed is:

1. An abuse deterrent pharmaceutical composition comprising:
a. a prodrug of a pharmaceutically active ingredient; and
b. an enzyme inhibitor;
wherein the enzyme inhibitor retards conversion of the prodrug to the pharmaceutically active ingredient when the composition is ingested in excess of an intended dosage.

2. The composition of claim 1, wherein the prodrug comprises a pharmaceutically active ingredient modified with a promoiety.

3. The composition of claim 2, wherein the promoiety modifies a ketone group of the pharmaceutically active ingredient.

4. The composition of claim 2, wherein the promoiety modifies a hydroxyl group of the pharmaceutically active ingredient.

5. The composition of claim 1, wherein the pharmaceutically active ingredient comprises a drug susceptible to abuse.

6. The composition of claim 1, wherein the enzyme inhibitor is configured to bind with an enzyme when the composition is ingested in excess of an intended dosage.

7. The composition of claim 6, wherein the enzyme is selected from the group consisting of:
CYP2D6, CYP3A4, CYP2D6, CYP2C8, CYP2C19, CYP2D6, and CYP2C9.

8. The composition of claim 6, wherein the enzyme is a digestive enzyme.

9. The composition of claim 1, wherein the enzyme inhibitor is configured to protect the promoiety from being removed by an enzyme when the composition is ingested in excess of an intended dosage.

10. The composition of claim 3, wherein the promoiety is attached to the pharmaceutically active ingredient by an enzymatically cleavable bond.

11. The composition of claim 10, wherein the enzyme inhibitor is configured to protect the enzymatically cleavable bond from being cleaved by an enzyme when the composition is ingested in excess of an intended dosage.

12. The composition of claim 9, wherein the enzyme is selected from the group consisting of:
CYP2D6, CYP3A4, CYP2D6, CYP2C8, CYP2C19, CYP2D6, and CYP2C9.

13. The composition of claim 9, wherein the enzyme is a digestive enzyme.

14. The composition of claim 1, wherein the pharmaceutically active ingredient is an opioid.

15. The composition of claim 1, wherein the prodrug is an ester of the pharmaceutically active ingredient.
